(19) United States
(12) Patent Application Publication
(10) Pub. No.: US 2026/0041379 A1
(43) Pub. Date: Feb. 12, 2026

(54) NON-INTRUSIVE, NEEDLE-FREE DETECTION OF THE SEVERE HYPOGLYCEMIA ONSET

(71) Applicants: Alexander Landau, Boulder, CO (US); Igor Landau, Boulder, CO (US)

(72) Inventors: Alexander Landau, Boulder, CO (US); Igor Landau, Boulder, CO (US)

(21) Appl. No.: 18/798,417

(22) Filed: Aug. 8, 2024

(51) Int. Cl. A61B 5/00

(52) U.S. Cl. CPC A61B 5/7282

(57) ABSTRACT

Method for non-intrusive, needle-free detection of severe hypoglycemia onset through assessment of the cerebral arteriovenous oxygen difference. The method primarily pertains to the treatment of high-risk younger patients with Type 1 Diabetes Mellitus (T1DM) experiencing nocturnal hypoglycemia in order to prevent so called “Death in Bed” syndrome-the sudden death of young diabetic patients without any history of long-term complications.

### FIELD OF THE INVENTION

The present invention primarily pertains to the treatment of high-risk younger patients with Type 1 Diabetes Mellitus (T1DM) experiencing hypoglycemia, a condition characterized by lower-than-normal blood glucose levels. Specifically, this invention relates to a completely non-intrusive, needle-free technique for the detection severe nocturnal hypoglycemia, which impairs normal brain neuronal activity, and, without immediate intervention, could result in the sudden death of young diabetic patients without any history of long-term complications.

### BACKGROUND OF THE INVENTION

The brain is one of the most energy-demanding and metabolically active organs in the body. Although it comprises only 2% of body weight, it receives about 15% of cardiac output. Glucose is the essential metabolic fuel for the brain, which uses 25% of total body glucose.

An acute and severe reduction in brain glucose quickly leads to impairment of cognitive and reflex functions. After severe hypoglycemic bouts, the brain's central mechanisms become less responsive to glucose deficits, resulting in “hypoglycemia unawareness” that leaves the individual physiologically defenseless against the development of hypoglycemia, greatly increasing the risk of death from a subsequent hypoglycemic episode.

In younger T1DM patients'hypoglycemia is the most significant and common side effect of insulin therapy and can pose a particular problem. Each year, about 25% of patients on intensive insulin therapy experience at least one episode of severe hypoglycemia.

Repeated episodes can impair the counter-regulatory system, potentially leading to hypoglycemia unawareness. Studies suggest that more than half of all severe hypoglycemia episodes occur at night during sleep. Nocturnal hypoglycemia is common among younger patients with Type 1 diabetes and can be potentially extremely dangerous. Timely detection of nocturnal severe hypoglycemia onset necessitates continuous glucose monitoring during the night.

Continuous Glucose Monitoring (CGM) devices currently used to evaluate nocturnal blood glucose levels are costly and have their own set of problems. These devices require a needle sensor inserted under the skin, which must be replaced every few weeks. The sensors are prone to high incidences of false alarms and are expensive.

Constructed from “biocompatible” materials, the needle sensors are quickly coated with a layer of protein, which can reduce their sensitivity over time, causing lower responses than the actual glucose levels. This effect limits the sensor's lifespan and may require frequent recalibration with a finger-stick meter. The sensor operates properly when bathed in interstitial fluid but can malfunction if firm tissue contact restricts access to this fluid, potentially generating false hypoglycemia alarms. At best, this effect limits the number of days a sensor will live in tissue and can require that the sensor be recalibrated at frequent intervals with a finger-stick meter.

As meticulously documented in John L. Smith's “The Pursuit of Noninvasive Glucose: ‘Hunting the Deceitful Turkey’,” non-invasive, needle-free continuous glucose monitoring has remained an elusive target for over 35 years. Therefore, new techniques are needed to overcome these disadvantages.

### SUMMARY OF THE INVENTION

The invention for needle-free detection of severe hypoglycemia onset exploits the fact that the dangerous decline of glucose transport to the brain cells consequently causes a matched decrease in the amount of oxygen in cerebral glucose metabolism. Such a decrease can be directly measured by the cerebral arteriovenous oxygen difference in a completely non-intrusive, needle-free way.

Utilization of the current, of the shelf, devises to monitor the oxygen levels in the brain's arterial and venous blood vessels, allowing for real-time detection of hypoglycemia without the need for invasive procedures. Such user-friendly and comfortable to use during sleep technique ensuring continuous monitoring without disrupting the patient's rest. Furthermore, it could be easy to integrate with existing mobile devices, providing data analysis and alerts through dedicated applications.

1. Technique for non-intrusive, needle-free detection of the severe hypoglycemia onset via direct measurement of the cerebral arteriovenous oxygen difference.
